(19) United States
(12) Patent Application Publication
(10) Pub. No.: US 2003/0032943 A1
(43) Pub. Date: Feb. 13, 2003

(54) BIOCOMPATIBLE, INJECTABLE AQUEOUS SOLUTION FOR USE IN ULTRASOUND ENERGY ASSISTED SURGERY

(76) Inventor: MORIS TOPAZ, KIBBUTZ RAMAT HAKOVESH (IL)

Correspondence Address: BERNHARD D SAXE, FOLEY & LARDNER, WASHINGTON HARBOUR, 3000 K STREET NW SUITE 500, WASHINGTON, DC, 200075109

(21) Appl. No.: 09/478,363

(22) Filed: Jan. 6, 2000

(30) Foreign Application Priority Data
Jan. 7, 1999 (IL) 127.967

(51) Int. Cl. A61M 37/00

(52) U.S. Cl. 604/542; 424/1.13

(57) ABSTRACT

The invention provides a biocompatible, injectable aqueous solution for use in high intensity ultrasound energy assisted surgery comprising a gas selected from the group consisting of carbon dioxide, nitrogen and mixtures thereof for the reduction and limitation of cavitation.

### FIELD OF INVENTION

The present invention relates to a biocompatible, injectable aqueous solution for use in high intensity ultrasound energy assisted surgery. More specifically the irrigation solution of the present invention contains a gas selected from the group consisting of carbon dioxide, nitrogen and mixtures thereof for the reduction and limitation of cavitation and the reduction and limitation of sonoluminescence and sonochemistry.

### BACKGROUND OF THE INVENTION

Ultrasound energy has been extensively used in the medical field, mainly for diagnostic and therapeutic purposes it has been utilized for diagnosis in medicine mainly at a sonar pulse mode of very short duration and of high frequency and peak intensity, or at low intensities of 10⁻² to 10^{-3 W/cm}² CW, and at a high frequency in dopier mode. Ultrasound is used in physical therapy in intensities of up to 3-5 W/Cm² CW, for a short exposure time. Ultrasonic ablative surgery instrumentation, such as those used in phacoemulsification in ophthalmology, recanalization and thrombus dissolution in peripheral arteries, in neurosurgery, hepatic, renal, prostate and bladder resections, as well as in extracorporeal shock wave lithotripter, utilize higher power intensities that are above 10W/cm²CW. The long-term safety of diagnostic ultrasonic equipment in clinical use was questioned and evaluated with no significant effects demonstrated at intensities below 100 mW/cm² spatial and temporal peak average intensity.

High-intensity ultrasound energy (HIUE) has been used in the last three decades in a wide range of surgical procedures for the extraction of tissues such as soft tissues and other target tissues, said target tissues including kidney tissues, brain tissues, liver tissues, cataracts, etc. In the last ten years, HIUE has been utilized for aesthetic surgery in Ultrasound-Assisted Lipoplasty (UAL). In UAL, subcutaneous fatty tissue in the human body is irrigated with wetting solution, emulsified by exposure to HIUE through a probe via a stab wound in the skin, and can be evacuated simultaneously or immediately following, by the application of low-vacuum suction.

The standard conditions for operating HIUE are usually at an intensity range above continuous wave (CW) 10W/cm², frequencies in the range of about 20-100 kHz, and amplitudes in the range of 60 (low)−320 (ultrahigh) micrometers, double (peak-to-peak) amplitude of the radiation face of the tip in micrometers on sonicator processor operating at maximum output.

In order to avoid the thermal effect resulting from the exposure of soft tissue to HIUE, a physiological water solution is injected into the subcutaneous tissue before and/or during irradiation, resulting in a substantial reduction in temperature rise, and peak temperature, thus avoiding thermal burn. The application of HIUE in an aqueous medium, however, has been proved to generate cavitation.

Cavitation is the expansion and rapid adiabatic collapse of tiny gas bubbles in solution, with energy sufficient to produce sonoluminescence and sonochemistry and temperature rise. Sonoluminescence is the conversion of sound energy into electromagnetic energy in the form of light in the visible, UV and possible soft x-ray range of the spectrum. Sonoluminescence is the direct indication of cavitation generated by the application of HIUE to aqueous solution. Sonochemistry is the generation of highly reactive chemical products such as free radicals and oxydizing reactants in the cavitation medium.

UV light and free radicals were found in previous scientific reports to lead to changes in biological systems, by alteration of the DNA sequence, changes in RNA, cell membranes, other cell infrastructures and in other macromolecules. A review article by the present inventor that appeared in the Aesthetic Surgery Journal 1998;18:19-24, “Possible Long-term Complications in Ultrasound-assisted Lipoplasty Induced by Sonoluminesence, Sonochemistry, and Thermal Effect” by the present inventor, as well as a letter to the editor “Long-Term Possible Hazardous Effects of Ultrasonically Assisted Lipoplasty” published in Plastic and Reconstructive Surgery, 1998;102:280, sparked controversy regarding possible long-term adverse effects of UAL. In further work, the present inventor has found that continuous wave (CW) HIUE applied under conditions that simulate UAL operating in an intercellular environment and current instrumentation, is above threshold intensities under which large quantities of free radicals and oxydizing reactants are produced, and in which sonoluminescence is generated. This irradiation may induce long-term risks for patients undergoing exposure to HIUE, particularly in long exposure, the exposure of sensitive tissue such as breast and nerve, and in young-age patients.

The use of scavengers in oral administration, such as vitamins A, C, D, E and others, was proposed prior to or after exposure to sonication in order to reduce the possible effects of free radicals. The limitation of the oral application of scavengers lies in the need for high concentrations of these substances to avoid the effects of free radicals. The addition the above substances to the sonicated medium does not inhibit cavitation with the subsequent generation of sonoluminescence and Sonochemistry.

A known method for eliminating cavitation is by degassing of the solution prior to the application of HIUE. The above method would be impractical in a common surgical procedure because gas would infiltrate into the wetting solution through the stab wounds in the skin to the subcutaneous tissue. It would be very hard to prevent gases from entering the tissue and solution through the surgical ports during the steps of injecting wetting solution, elevation or retraction of the skin prior or during sonication. All of the above steps would allow gas to reenter the subcutaneous tissue and the sonicated solution.

The application of HIUE to tissue leads to fragmentation in a complicated mechanism. The mechanical, longitudinal action of the probe creates a shearing effect which leads to a sharp and drastic elevation in pressure and temperature and thus damage or destruction of tissue structures cell membrane, and other infrastructures. The addition of water to the biological environment would actually significantly reduce the rate of temperature elevation and the peak temperature, yet would lead to cavitation that would eventually lead to the formation of sonochemistry and sonoluminescence. As is realized from the above, there exists a need to eliminate cavitation during the sonication of tissue.

### SUMMARY OF INVENTION

Thus the present invention now provides a biocompatible, injectable aqueous solution for use in high intensity ultrasound energy assisted surgery comprising a gas selected from the group consisting of carbon dioxide, nitrogen and mixtures thereof for the reduction and limitation of cavitation.

In a preferred embodiment of the present invention there is provided a solution for use in ultrasound assisted lipoplasty comprising carbon dioxide for the reduction and limitation of cavitation.

In another aspect of the present invention there is provided a method for reducing and limiting cavitation in ultrasound assisted lipoplasty comprising, irrigating an area of target fatty tissue with an irrigating solution, inserting a probe into said area, ultrasonically vibrating said probe at standard conditions creating localized tissue separation and frictional heat; emulsification of at least some of said fatty tissue by said mechanical and sonic vibrations, so as to provide safer removal of said fatty tissue, emulsifying said fatty tissue; and evacuating the emulsified fatty tissue by applying suction, characterized in that said irrigating solution further comprises a gas selected from the group consisting of carbon dioxide, nitrogen and mixtures thereof.

In a preferred embodiment of the present invention there is provided a method for removing target tissue from a patient comprising: irrigating an area of target tissue with an irrigating solution, inserting a probe into said area of target tissue, ultrasonically vibrating said probe at standard conditions creating localized tissue separation and frictional heat; emulsifying at least some of said target tissue by said mechanical and sonic vibrations, so as to provide safer removal of said target tissue, emulsifying said target tissue; and evacuating the emulsified target tissue by applying suction, characterized in that a gas selected from the group consisting of carbon dioxide, nitrogen and mixtures thereof is introduced into said irrigating solution in the area exposed to high intensity ultrasound energy.

An even more preferred embodiment of the present invention provides a method for removing fatty tissue from a patient comprising: irrigating an area of target fatty tissue with an irrigating solution, inserting a probe into said area of fatty tissue; ultrasonically vibrating said probe at standard conditions creating localized tissue separation and frictional heat; emulsifying at least some of said fatty tissue by said mechanical and sonic vibrations, so as to provide safer removal of said fatty tissue, emulsifying said fatty tissue; and evacuating the emulsified fatty tissue by applying suction, characterized in that a gas selected from the group consisting of carbon dioxide, nitrogen and mixtures thereof is introduced into said irrigating solution in the area sonicated by said probe.

In especially preferred embodiment there is provided a method for removing target tissue from a patient wherein said gas is present in an amount sufficient to reduce cavitation in the area of sonication.

In an even further preferred embodiment the method of the present invention further comprises introducing a water soluble scavenger into said irrigating solution wherein said water soluble scavenger is vitamin C or a source thereof.

In the most preferred embodiments of the present invention said gas is carbon dioxide, however as will be realized the carbon dioxide used for suppressing cavitation, sonoluminescence and sonochemistry according to the present invention can be used in combination with other acceptable gases such as nitrogen.

The invention will now be described in connection with certain preferred embodiments with reference to the following illustrative figures so that it may be more fully understood.

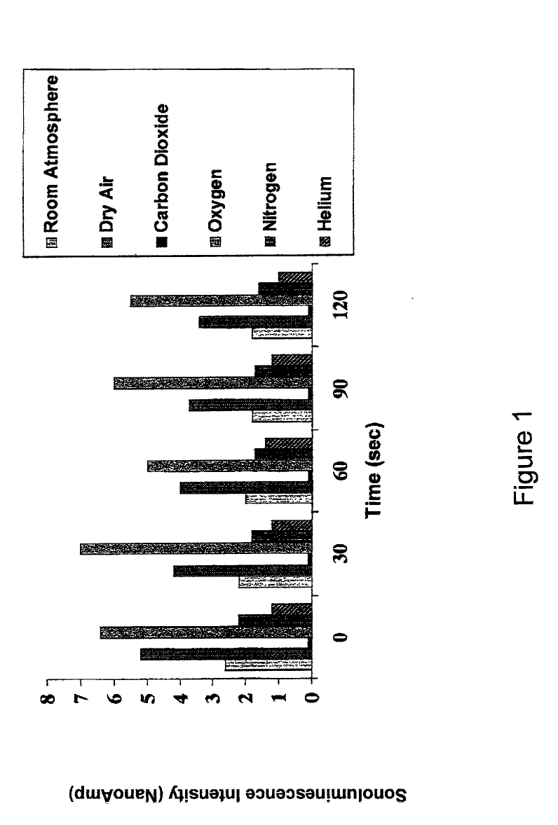

FIG. 1 is a comparative graphical representation of the influence of various gases on sonoluminescence generated by HIUE.

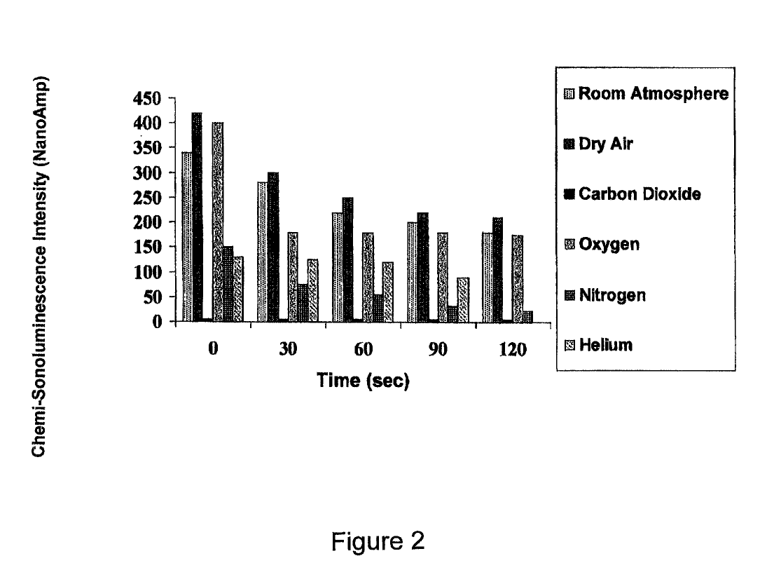

FIG. 2 is a comparative graphical representation of the influence of various gases on chemi-sonoluminescence.

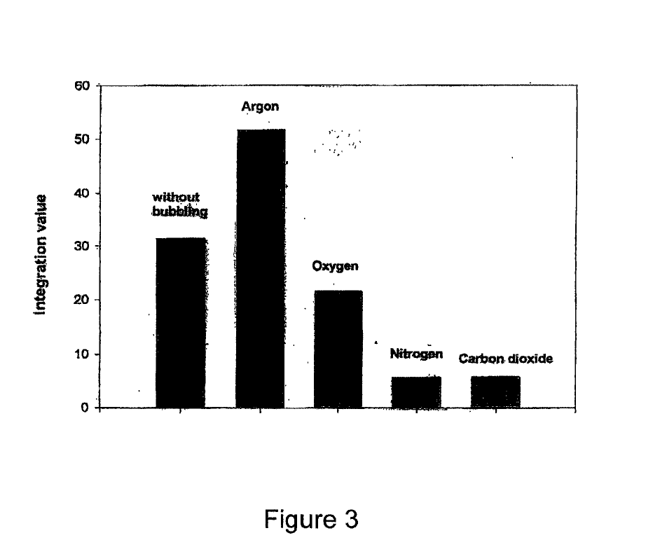

FIG. 3 is a comparative graphical representation of the influence of various gases on the formation of hydroxyl free-radicals.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 illustrates values of Sonoluminescence that were detected by sensitive photomultiplication technique. Sonoluminescence was generated by the cavitation phenomenon by sonication of aqueous physiological solution with HIUE in the presence of various gases. The gas flow was 100 ml/min in a saturated saline solution in order to determine the effect each gas had on Sonoluminescence. As can be seen the presence of carbon dioxide nearly eliminated Sonoluminescence thereby eliminating the creation of ultra violet light and possible soft x-ray radiation, while the use of nitrogen substantially reduced the same.

FIG. 2 illustrates values of Chemi-Sonoluminescence that were detected by sensitive photomultiplication technique. Chemi-Sonoluminescence was generated by the cavitation phenomenon in presence of Luminol (200 micl 10⁻² M Luminol, 50% Intensity amplitude) by sonication of aqueous physiological solution with HIUE in the presence of various gases. The gas flow was 100 ml/min in a saturated saline solution in order to determine the effect each gas had on Chemi-sonoluminescence. As can be seen the presence of various gases resulted in varying intensities of Chemi-sonoluminescence depending upon the effect of said gases on cavitation.

FIG. 3 illustrates the relative Integration values of an electron spin resonance (ESR) spectra of the DMPO-OH spin adduct signal peaks generated by sonication of solution which contained saline and each of the gases mentioned within the figure. The sonication conditions were 20sec., 3.4 relative intensity. As can be seen the presence of carbon dioxide significantly reduced the generation of hydroxy free radicals, as did the presence of nitrogen.

It will be evident to those skilled in the art that the invention is not limited to the details of the foregoing illustrative embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A biocompatible, injectable aqueous solution for use in high intensity ultrasound energy assisted surgery comprising a gas selected from the group consisting of carbon dioxide, nitrogen and mixtures thereof for the reduction and limitation of cavitation.

2. A solution according to claim 1 for use in ultrasound assisted lipoplasty comprising a gas selected from the group consisting of carbon dioxide, nitrogen and mixtures thereof for the reduction and limitation of cavitation.

3. A solution according to claim 1, wherein said gas is carbon dioxide.

4. A method for reducing and limiting cavitation in ultrasound assisted lipoplasty comprising, irrigating an area of target fatty tissue with an irrigating solution, inserting a probe into said area of fatty tissue; ultrasonically vibrating said probe at standard conditions creating localized tissue separation and frictional heat; emulsification of at least some of said fatty tissue by mechanical and sonic vibrations, so as to provide for removal of said fatty tissue; emulsifying said fatty tissue; and aspirating the emulsified fatty tissue by applying suction, characterized in that said irrigating solution further comprises a gas selected from the group consisting of carbon dioxide, nitrogen and mixtures thereof.

5. A method for removing target tissue from a patient comprising: irrigating an area of target tissue with an irrigating solution, inserting a probe into said area of target tissue; ultrasonically vibrating said probe at standard conditions creating localized tissue separation and frictional heat; emulsifying at least some of said target tissue by mechanical and sonic vibrations, so as to provide for removal of said target tissue; emulsifying said target tissue; and evacuating the emulsified target tissue by applying suction, characterized in that a gas selected from the group consisting of carbon dioxide, nitrogen and mixtures thereof is introduced into said irrigating solution in the area exposed to high intensity ultrasound energy.

6. A method for removing fatty tissue from a patient comprising: irrigating an area of target fatty tissue with an irrigating solution, inserting a probe into said area of fatty tissue; ultrasonically vibrating said probe at standard conditions creating localized tissue separation and frictional heat; emulsifying at least some of said fatty tissue by mechanical and sonic vibrations, so as to provide for removal of said fatty tissue, emulsifying said fatty tissue; and evacuating the emulsified fatty tissue by applying suction, characterized in that a gas selected from the group consisting of carbon dioxide, nitrogen and mixtures thereof is introduced into said irrigating solution in the area sonicated by said probe.

7. A method for removing fatty tissue from a patient according to claim 6 wherein said gas is present in an amount sufficient to reduce cavitation in the area of sonication.

8. A method according to claim 4, further comprising introducing a water soluble scavenger into said irrigating solution.

9. A method according to claim 8 wherein said water soluble scavenger is vitamin C.

10. A method according to claim 4, wherein said gas is carbon dioxide.

11. A method according to claim 5, wherein said gas is carbon dioxide.

12. A method according to claim 6, wherein said gas is carbon dioxide.
